(19) United States
(12) Patent Application Publication
(10) Pub. No.: US 2013/0103086 A1
(43) Pub. Date: Apr. 25, 2013

(54) SPINOUS PROCESS MOUNTED SPINAL IMPLANT

(71) Applicants: Gregory Marik, Collierville, TN (US); Samuel A. Pumphrey, Hernando, MS (US); Matthew J. Van Nortwick, Memphis, TN (US)

(72) Inventors: Gregory Marik, Collierville, TN (US); Samuel A. Pumphrey, Hernando, MS (US); Matthew J. Van Nortwick, Memphis, TN (US)

(73) Assignee: Warsaw Orthopedic, Inc., Warsaw, IN (US)

(21) Appl. No.: 13/277,012

(22) Filed: Oct. 19, 2011

(51) Int. Cl. A61B 17/70; A61B 17/88

(52) U.S. Cl. 606/246; 606/279

(57) ABSTRACT

A spinal implant includes two plates that are connected together by a post. The implant is configured for each plate to be positioned on outer lateral sides of spinous processes with the post extending through the interspinous space. One of the plates includes a channel that receives the post. The channel is configured so that the plate can be assembled over the post from a direction normal to the post's axis. This configuration allows the plate to be added in a posterior to anterior direction for the typical situation, without having to first locate the plate laterally outboard of the post's tip. As such, the spinal implant allows for a more compact surgical site to be used for the spinal implant implantation procedure.

### BACKGROUND

The present invention generally relates to devices and methods for stabilizing vertebral members, and more particularly, to spinal implants that mount onto the spinous processes.

Vertebral members typically comprise a vertebral body, pedicles, laminae, and processes. The processes are projections that serve as connection points for the ligaments and tendons, and typically include the articular processes, transverse processes, and the spinous process. Intervertebral discs are located between adjacent vertebral bodies to permit flexion, extension, lateral bending, and rotation.

Various conditions may lead to damage of the intervertebral discs and/or the vertebral members. The damage may result from a variety of causes including a specific event such as trauma, a degenerative condition, a tumor, or infection. Damage to the intervertebral discs and vertebral members can lead to pain, neurological deficit, and/or loss of motion. One manner of correcting the damage involves mounting of a spinal implant onto the spinous processes, typically in association with a fixation process such as anterior lumbar interbody fusion (ALIF), posterior lumbar interbody fusion (PLIF), intertransverse lumbar interbody fusion (ILIF), and the like. See, for example, the spinal implant sold under the trade name CD HORIZON SPIRE™ by Medtronic Spinal and Biologics of Memphis, Tenn., and the devices described in U.S. Patent Application Publication 2006/0247640 and U.S. Pat. No. 7,048,736. While these devices provide some solutions, they may not be ideal for some situations. As such, there remains a need for alternative spinal implants and related methods.

### SUMMARY

A spinal implant includes with two plates that are connected together by a post. The implant is configured for each plate to be positioned on outer lateral sides of spinous processes with the post extending through the interspinous space. One or both plates may be movable along the length of the post to accommodate different anatomies such as for relatively wide or thin spinous processes. One of the plates includes a channel that receives the post. The channel is configured so that the plate can be inserted over the post from a direction normal to the post's axis. This configuration allows the plate to be added in a posterior to anterior direction for the typical situation, without having to first locate the plate laterally outboard of the post's tip. As such, the spinal implant allows for a more compact surgical site to be used for the spinal implant implantation procedure.

In one aspect, art embodiment of the present invention provides a spinal implant comprising a first plate, a second plate disposed in spaced relation to said first plate, and a post. The first plate has a medial face configured to confront adjacent spinous processes. The second plate has a medial face configured to confront the adjacent spinous processes. The post connects the second plate to the first plate and extends distally from the first plate to a post distal tip along a longitudinal post axis oriented transverse to the medial face of the second plate. The medial faces of each of the plates extend from the post in opposite directions therefrom between a superior end section of the respective plate that is positionable along a superiorly located spinous process and an inferior end section of the respective plate positionable along an inferiorly located spinous process. The medial faces of both the first and second plates in both of their superior and inferior end sections each have a plurality of protrusions configured to bite into the corresponding spinous process. The second plate has a fastener bore and an anterior edge disposed opposite the fastener bore. The second plate has a channel therein opening at the anterior edge and extending posteriorly generally toward the fastener bore. The channel has a locking surface disposed posteriorly of the anterior edge and in spaced relation to the fastener bore. The fastener bore opens anteriorly to the channel. The channel and the post are jointly configured such that the second plate may be moved over the post in an anterior direction normal to the post axis, while the post extends distally of the medial face of the second plate, to move the post from a position external to the second plate, posteriorly past the anterior edge of second plate, and into engagement with the locking surface. The fastener, post, and second plate are configured such that anterior advancement of the fastener, while the post abuts the locking surface, clamps the post between the fastener and the locking surface.

In another aspect, an embodiment of the present invention provides a spinal implant having a first plate, a second plate disposed in spaced relation to said first plate and having a channel, a post, and a fastener. The first plate has a medial face configured to confront adjacent spinous processes. The second plate has a medial face configured to confront the adjacent spinous processes. The post connects the second plate to the first plate and extends distally from the first plate to a distal tip along a longitudinal post axis oriented transverse to the medial face of the second plate. The medial faces of each of the plates extend from the post in opposite directions therefrom between a superior end section of the respective plate that is positionable along a superiorly located spinous process and an inferior end section of the respective plate positionable along an inferiorly located spinous process. The medial faces of both the first and second plates in both of their superior and inferior end sections each have a plurality of protrusions configured to bite into the corresponding spinous process. The second plate has a fastener bore and an anterior edge opposite the fastener bore. The second plate has a channel therein defined by a boundary wall. The channel opens at the anterior edge and extends posteriorly generally toward the fastener bore. A portion of the boundary wall forms a posteriorly facing locking surface. The fastener is disposed in the fastener bore and is moveable relative to the second plate in a fastener advancement direction. A first theoretical plane is disposed normal to the post axis. The post, from the medial face of the second plate to its distal tip, has a cross-section having a first size projected onto the first plane. The post is movable relative to the second plate, while extending through the channel such that the post tip is distally disposed relative to the medial face of the second plate and a centerline of the fastener bore, between a load position and a lock position. The implant is configured such that, with the post in the load position: a) the post is spaced from the locking surface; b) superior and inferior surfaces of the post directly face the boundary wall of the channel in unobstructed fashion; c) a cross-section of the channel from the post to the anterior edge, projected onto the theoretical plane, does not narrow to less than the first size of the post. The implant is also configured such that, with the post in the lock position: a) the fastener supplies a force to clamp the post against the locking surface to affix the second plate to the post.

In another aspect, the present invention provides a method of implanting a spinal implant. The method includes positioning a first plate along a first lateral side of adjacent spinous processes, with the positioning comprising disposing a post mounted to the first plate through the interspinous space between the adjacent spinous processes. The method also includes positioning a second plate posteriorly of the first plate with a distal tip of the post disposed distally of the medial face of the second plate. The second plate has a fastener bore and a channel therein disposed anteriorly of the fastener bore. The channel extends from an anterior edge of the second plate generally toward the fastener bore and includes a locking surface. The method further includes thereafter moving the second plate anteriorly relative to the post so that the post moves past the anterior edge and into the channel. The method subsequently includes a) moving the second plate further anteriorly relative to the post so that the post abuts the locking surface; b) moving the second plate along the post toward the first plate so that protrusions on medial faces of both the first and second plate bite into the adjacent spinous processes; c) thereafter, tightening a locking member disposed in the fastener bore to supply a clamping force that clamps the post against the locking surface. The method may further comprise pivoting the post relative to the first plate. The distal tip of the post may be disposed distally of the second plate during the moving the second plate anteriorly relative to the post so that the post moves past the anterior edge and into the channel. The moving the second plate so that the post abuts the locking surface may comprise moving the second plate both anteriorly and superiorly relative to the post. The tightening of the locking member may comprise advancing the locking member anteriorly by rotating the locking member relative to the second plate. At the conclusion of the tightening, the entirety of the post disposed distally of the medial face of the second plate is advantageously disposed posteriorly of the locking surface.

Some or all of the embodiments may have the following aspects. The fastener may be a setscrew. The fastener bore may have a centerline, with the centerline not passing through the locking surface. The post may be pivotally mounted to the first plate. The second plate is advantageously infinitely positionable along the post axis relative to the first plate. The centerline of the fastener bore advantageously does not pass through the locking surface.

Of course, the present invention is not limited to the above features and advantages. Indeed, those skilled in the art will recognize additional features and advantages upon reading the following detailed description, and upon viewing the accompanying drawings.

### BRIEF DESCRIPTION OF THE DRAWINGS

FIG. 1 shows a spinal implant according to one embodiment mounted to a spinal column.

FIG. 2 shows a perspective view of a spinal implant according to one embodiment.

FIG. 3 shows a partially exploded view of the implant of FIG. 2.

FIG. 4 shows a top (posterior to anterior) view of the implant of FIG. 2.

FIG. 5 shows a side view of the implant of FIG. 2, looking inward at the second plate.

FIG. 6 shows an end view of the implant of FIG. 2 during the assembly process, with the second plate not yet mounted to the post.

FIG. 7 shows an end view of the implant of FIG. 2 during the assembly process, with the post in the channel, in the loading position.

FIG. 8 shows an end view of the implant of FIG. 2 during the assembly process, with the post engaging the locking surface of the channel.

FIG. 9 shows an end view of the implant of FIG. 2 during the assembly process, with the second plate displaced toward the first plate and the fastener tightened to lock the second plate relative to the post.

### DETAILED DESCRIPTION

In one embodiment, the present application is directed to a spinal implant with two plates that are connected together by a post. The implant is configured for each plate to be positioned on outer lateral sides of spinous processes with the post extending through the interspinous space. One or both plates may be movable along the length of the post to accommodate different anatomies such as for relatively wide or thin spinous processes. One of the plates includes a channel that receives the post. The channel is configured so that the plate can be inserted over the post from a direction normal to the post's axis. This configuration allows the plate to be added in a posterior to anterior direction for the typical situation, without having to first locate the plate laterally outboard of the post's tip. As such, the spinal implant allows for a more compact surgical site to be used for the spinal implant implantation procedure.

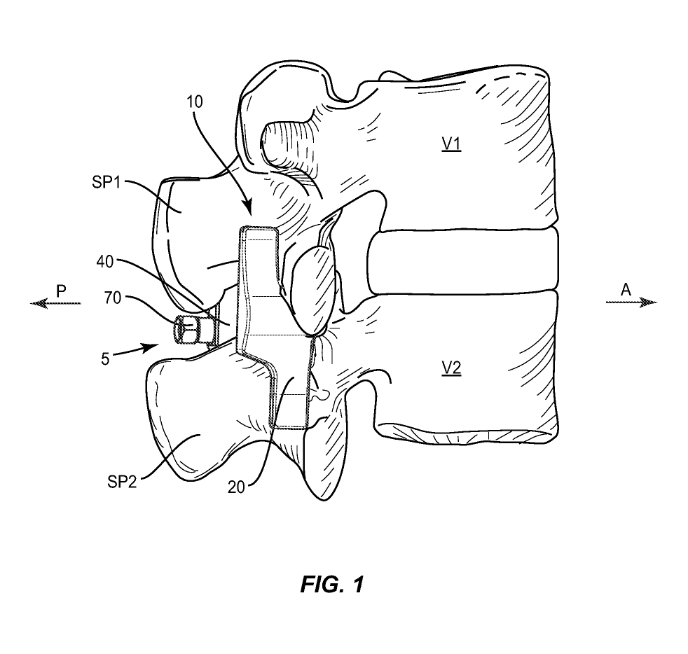

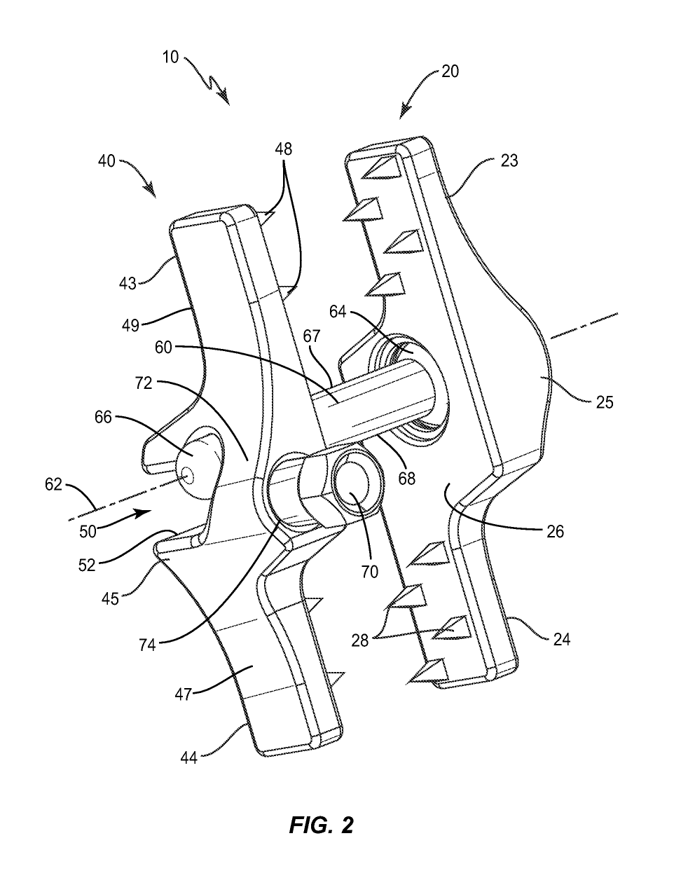

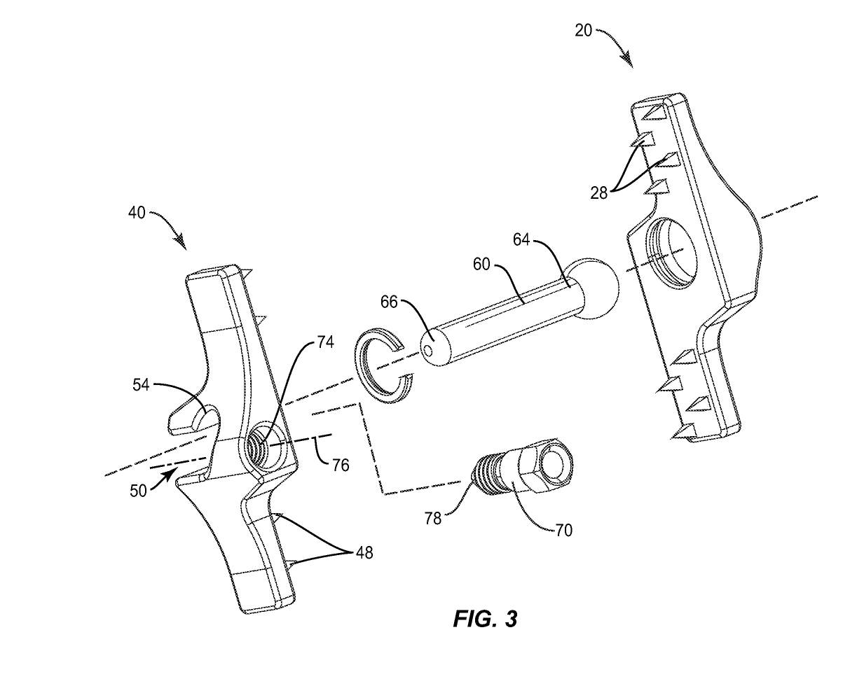

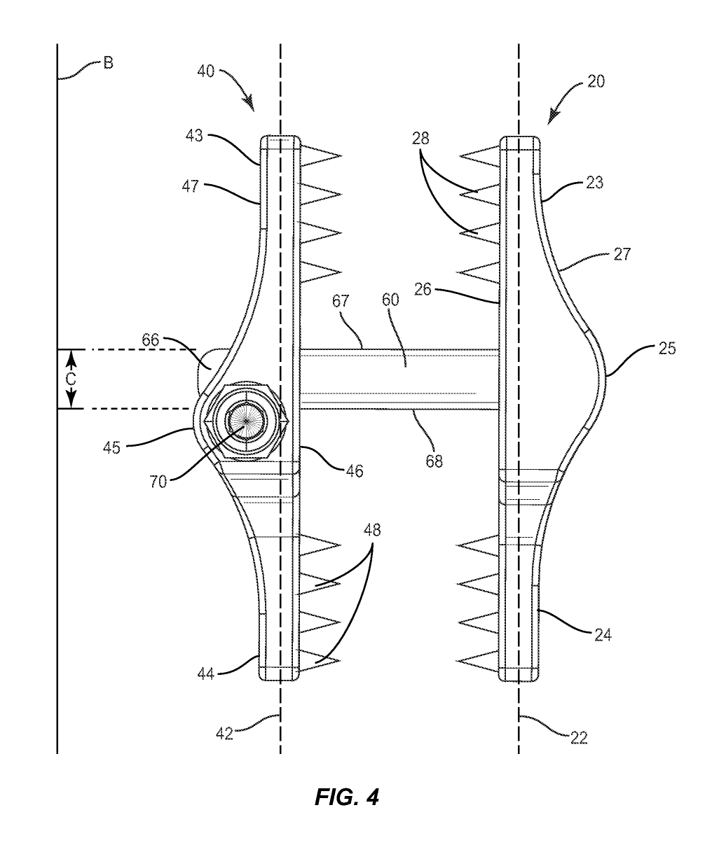

Referring to FIG. 1, a spinal implant according to one embodiment and generally designated 10 is shown clampingly mounted to the spinous process SP1 of a superior vertebra V1 and a spinous process SP2 of an inferior vertebra V2. A portion of the implant 10, in particular post 60, extends transversely through the interspinous space 5 between the two spinous processes SP1,SP2. The implant, shown more clearly in FIGS. 2-5, includes a first plate 20, a second plate 40, an interconnecting post 60, and a fastener 70.

The first plate 20 may be elongate along an associated longitudinal axis 22, with a superior end section 23, an inferior end section 24, and an intermediate section 25. If desired, the superior end section 23 and inferior end section 24 may be shifted in an anterior direction A or a posterior direction P so that the first plate has a somewhat Z-shape as shown, although this is optional and the first plate 20 may be generally rectilinear or any other suitable shape as is desired. The first plate 20 has a length sufficient to vertically span the interspinous gap 5 (interspinous space) between adjacent spinous processes while substantially overlapping the spinous processes SP1,SP2. The first plate 20 has a medial face 26 and an opposite lateral face 27. The medial face 26 includes a plurality of protrusions 28 that extend medially for biting into the corresponding spinous process SP1,SP2. Advantageously, the protrusions take the form of a plurality of sharp teeth. The teeth 28 may advantageously be disposed in two groups, one on the superior end section 23 and one on the inferior end section 24, with the intermediate section 25 being free of such teeth 28. The lateral face 27 may have suitable features, such as recesses or the like, for cooperating with installation and manipulation instrumentation. The tips of superior end 23 and inferior end 24 are advantageously generally rounded so as to minimize damage to surrounding tissue and for ease of installation.

The second plate 40 may be substantially similar to the first plate 20. For example, the second plate 40 may be elongate along an associated longitudinal axis 42, with a superior end section 43, an inferior end section 44, and an intermediate section 45. If desired, the second plate 40 may have a somewhat Z-shape similar to the first plate 20, or may be any other suitable shape as is desired. The second plate 40 advantageously has a length sufficient to vertically span the interspinous gap 5 while substantially overlapping the spinous processes SP1,SP2. The second plate 40 has a medial face 46 and an opposite lateral face 47, with the medial face 46 facing the medial face 26 of the first plate 20. The medial face 46 includes a plurality of protrusions 48 similar to teeth 28 for biting into the spinous processes SP1,SP2. The lateral face 47 may have suitable features, such as recesses or the like, for cooperating with installation and manipulation instrumentation. The tips of superior end section 43 and inferior end section 44 are advantageously generally rounded so as to minimize damage to surrounding tissue and for ease of installation. The intermediate section 45 may have a suitable boss 72 thereon, with a hole 74 for receiving the fastener 70, as discussed below. Anterior edge 49 of second plate 40 is disposed generally opposite hole 74. The intermediate section 45 of the second plate 40 includes a channel 50 that extends generally perpendicular to the longitudinal axis 42 toward anterior edge 49 and is sized to receive post 60, as discussed further below.

The post 60 of FIG. 2 takes the form of a simple round shaft that extends along a post axis 62 from a proximal end 64 to a distal tip 66. The post 60 has a length sufficient to extend laterally across the interspinous gap, through the medial face 46 of the second plate 40, and into engagement with the fastener 70. In some embodiments, the post proximal end 62 is mounted to the first plate 20 by any suitable means, such as welding or the like. Alternatively, the post 60 may be integrally formed with the first plate 20. Still further, the post 60 may be pivotally attached to the first plate 20. For example, the post 60 may be pivotally attached either for monoaxial or polyaxial movement relative to the first plate 20 about one or more pivot axes, such as about a pivot axis perpendicular to the post axis 62 and parallel to medial face 26. Examples of suitable pivoting structures are shown in U.S. Pat. No. 7,048,736 and U.S. Patent Application Publications 2006/0247640. The post 60 may include flats or other features (not shown) for engaging with the fastener 70. The post 60 may advantageously be solid rather than hollow, and therefore typically will not have any longitudinal hollow inner cavities. The post 60 may have a cylindrical distal extent, or may have other convenient cross-sectional shapes, such as rectangular, hexagonal, oval, and the like.

The fastener or locking member 70 of FIG. 2 takes the form of a simple setscrew with tapered tip 78 that is sized to threadably engage hole 74 in second plate 40. When tightened, the locking member 70 presses against post 60 to lock the relative distance between the plates 20,40. Of course, other forms of fasteners, such as concentrically barbed posts, quarter-turn fasteners, and the like, may alternatively be used.

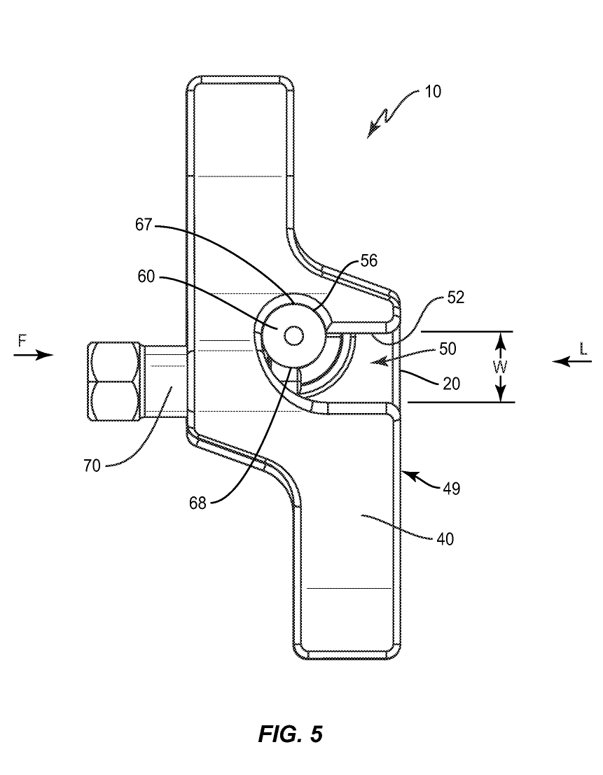

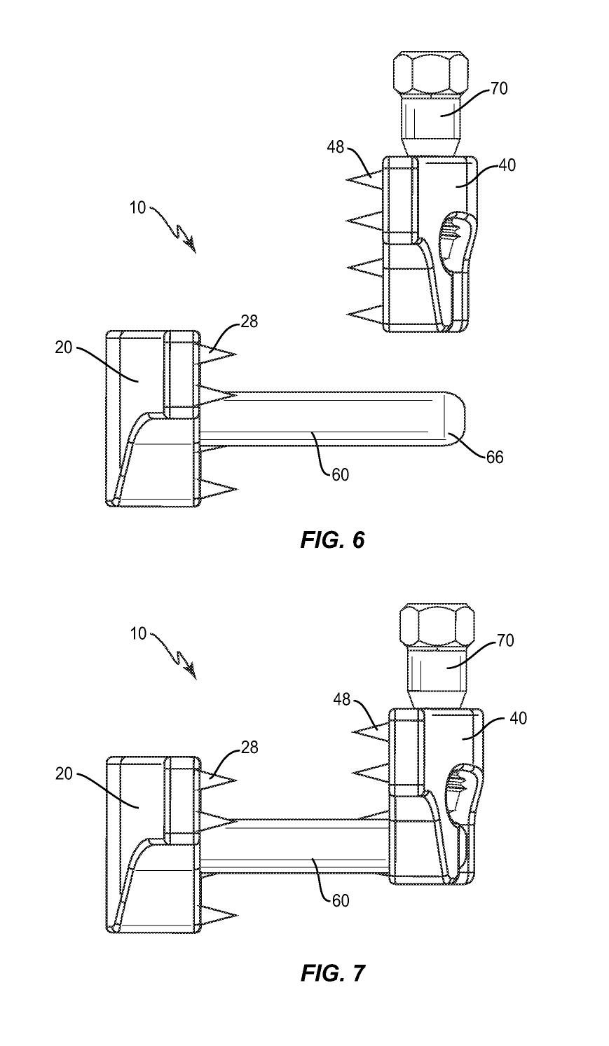

In order that the second plate 40 may be placed directly over the post 60 by moving in a posterior to anterior direction, and without having to first be located laterally outboard of the post 60, the second plate 40 includes channel 50. The channel 50 of FIG. 2 is oriented generally in an anterior to posterior direction. The channel 50 is bounded by a boundary wall 52 and opens to anterior edge 49 of second plate 40. Channel 50 includes a locking surface 54 disposed in spaced relation from anterior edge 49, toward hole 74 but spaced therefrom. As shown in FIG. 5, channel 50 may advantageously be somewhat L-shaped or J-shaped so that locking surface 54 is offset from hole 74 (which extends to channel 50) such that fastener bore centerline 76 does not pass through locking surface 54. As can be appreciated, locking surface 54 is oriented generally in a posterior direction, and thus is transverse to the main axis of channel 50. This orientation allows locking surface 54 to act as a clamping surface when fastener 70 is tightened against post 60.

The channel 50 has a width W that is wide enough that post 60 may move posteriorly therealong from a position outside of second plate 40, past the anterior edge 49, and to the locking surface 54. Thus, the post loading direction L into channel 50 is from anterior edge 49 toward hole 74. For the second plate 40 shown in FIG. 2, the loading direction L is normal to a theoretical plane (not shown) defined by post axis 62 and longitudinal axis 42 of second plate 40, although posterior directions at a non-normal acute angle relative to the plane may alternatively be employed. The channel 50 is wide enough from its entrance at anterior edge 49 to the locking surface 54 so that post 60 may move to locking surface 54 without interference. Thus, the projected size of channel 50, from a loading position (FIG. 7) to the anterior edge 49 is larger than the projected size of post 60. This can be seen with reference to plane of projection B, which is disposed normal to post axis 62 and disposed laterally outboard of the post tip 66. This plane B is parallel to the plane of the paper in FIG. 5. When projected onto plane B, the post 60 has a cross-sectional size C, while channel 50 has a projected width W from the post 60 to the anterior edge. This width W is at least as large as C, and advantageously slightly larger, so that there is not interference to insertion of post 60 in a posterior direction past the anterior edge 49. This width is advantageously maintained throughout the channel 50 to the locking surface 54.

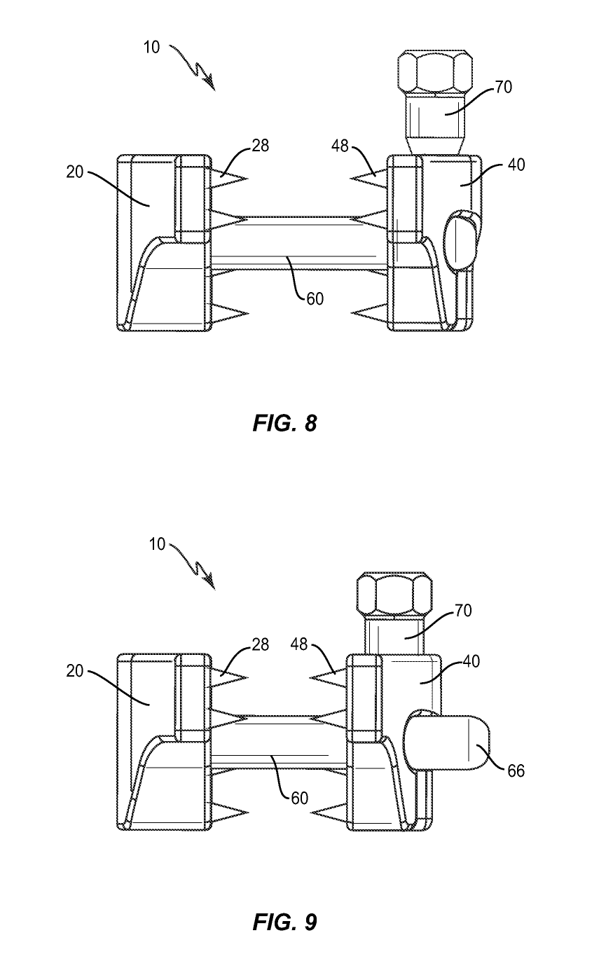

In use, the device 10 can be implanted for posterior spinal stabilization as a stand-alone procedure or in conjunction with other procedures. The device 10 can be positioned through a small posterior incision in the patient of sufficient size to admit the device and instrumentation. Following the incision, muscle is moved aside if and as needed for placement of the device 10. The spinous processes SP1,SP2 are optionally distracted using suitable instrumentation known in the art, and the first plate 20 is implanted such that the superior end section 23 extends on a first lateral side of spinous process SP1, inferior end section 24 extends on the first side of spinous process SP2, and post 60 extends through the interspinous space 5 generally normal to the sagittal plane defined by the spinous processes SP1,SP2. Note that the distal tip 66 of post 60 extends laterally beyond the spinous processes SP1,SP2 on the lateral side opposite first plate 20. The second plate 40 may then be added by positioning the second plate generally parallel to first plate 20, but slightly posterior thereto, with channel aligned with (but slightly anterior to) channel 50. Note that post distal tip 66 need not be positioned laterally outside of the second plate 40 (i.e., extending laterally beyond the lateral face 47 of second plate 40, opposite first plate 20), but may instead be disposed laterally even with lateral face 47 of second plate 40, or between lateral face 47 and medial face 46. The second plate 40 is then advanced in anterior direction A, resulting in post moving relative to second plate 40 in loading direction L (which is typically the same as posterior direction P) so that post 60 moves beyond the anterior edge 49 and into channel 50, but is still spaced from locking surface 54. See FIG. 7. This may be referred to as the load or loading position. As can be seen in FIG. 7, in the load position, the convex superior face 67 and convex inferior face 68 of post 60 both directly face the boundary wall 52 of channel 50 in an unobstructed fashion. The second plate 40 is then further advanced anteriorly and then slightly superiorly so that post 60 comes to rest against locking surface 54. See FIG. 8. The plates 20,40 are pushed toward one another with a compression instrument or manually, to move (e.g., slide) the second plate 40 along the post 60 toward the first plate 20 from a first position farther from the first plate 20 to a second position closer to the first plate 20. This movement causes the plates 20,40 to clamp the spinous processes SP1,SP2, with the spikes 28,48 biting into the bony material of the spinous processes SP1,SP2. Note that the sequence of abutting the post 60 against the locking surface 54 and the moving the plates 20,40 closer together may be reversed if desired. Locking member 70 is then tightened onto post 60 using an appropriate instrument to lock the relative positions of the plates 20,40. See FIG. 9. As can be seen, when fastener is advanced in fastener advancement direction F (which is typically the same as anterior direction A), tip 78 or other portion of fastener 70 presses against post 60, advantageously pressing post 60 anteriorly and inferiorly, thereby clamping post 60 between the fastener 70 and the locking surface 54. If desired, locking member 70 may be provided with a break-off portion (not shown) that provides an indication when sufficient torque is applied. Note that the second plate 40 is advantageously positionable along post 60 at an infinite number of positions, as the second plate 40 can conceptually slide to any number of positions along post 60 and be locked in the selected position by fastener 70.

While the above description has been in the context of an in-situ assembly of the first and second plates 20,40, in some embodiments, the device 10 may be inserted in an already-assembled condition, with the second plate 40 disposed on the post 60, with the device being either assembled by the manufacturer or by medical personnel.

Clamping plates 20,40 to the spinous processes SP1,SP2 helps maintain the alignment and spacing of the spinous processes SP1,SP2 while also providing resistance to spinal extension and flexion. Thus, engagement of plates 20,40 to the spinous processes SP1,SP2 resists movement of the spinous processes SP1,SP2 toward and away from one another as a result of spinal extension and flexion, respectively, or as a result of any other movement or condition.

As will be appreciated, the first plate 20, second plate 40, post 60, and fastener 70 may each be made from any suitable biocompatible rigid materials such as titanium and its alloys, stainless steel, cobalt chrome, ceramics, relatively rigid polymers like carbon reinforced polyetheretherketone (PEEK), or the like, known in the art. As can be appreciated, the first plate 20, second plate 40, and fastener 70 are advantageously distinct (e.g., separate) pieces from each other that are joined together during assembly.

In some embodiments, a sleeve (not shown) may be disposed on post to provide additional support of the vertebrae to maintain or provide post-operative distraction between the spinous processes SP1,SP2. The sleeve may be osteoconductive if desired. For more information on sleeves, see U.S. Patent Application Publication 2006/0247640.

The post 60 may have multiple longitudinal sections, with the distal tip 66 being on a removable section that is optionally removed once the second plate 40 is locked in place, see U.S. patent application Ser. No. 12/916,745, filed 1 Nov. 2010 and entitled “Spinous Process Implant with Extended Post.” Additionally or alternatively, the post 60 may have an enlarged distal tip 66, see U.S. patent application Ser. No. 12/916,761, filed 1 Nov. 2010 and entitled “Spinous Process Implant with a Post and an Enlarged Boss,”

While FIG. 1 shows an implant applied to vertebra L-4 and L-5, the implant device can be implanted on spinous processes at other levels. Levels up to T-3 may be appropriate sites. Also, plates 20,40 bridging more than one level may also be considered, optionally with multiple posts 60 disposed at suitable intervals.

The implant 10 may be used during surgical procedures on living patients. The implant may also be used in a non-living situation, such as within a cadaver, model, and the like. The non-living situation may be for one or more of testing, training, and demonstration purposes.

All U.S. patents, patent application publications, and applications mentioned above are hereby incorporated herein by reference in their entirety.

The present invention may, of course, be carried out in other ways than those specifically set forth herein without departing from essential characteristics of the invention. The present embodiments are to be considered in all respects as illustrative and not restrictive, and all changes coming within the meaning and equivalency range of the appended claims are intended to be embraced therein.

What is claimed is:

1. A spinal implant, comprising:
a first plate having a medial face configured to confront adjacent spinous processes;
a second plate having a medial face configured to confront the adjacent spinous processes; said second plate disposed in spaced relation to said first plate;
a post connecting the second plate to the first plate and extending distally from the first plate to a post distal tip along a longitudinal post axis oriented transverse to the medial face of the second plate;
wherein the medial faces of each of the plates extend from the post in opposite directions therefrom between a superior end section of the respective plate that is positionable along a superiorly located spinous process and an inferior end section of the respective plate positionable along an inferiorly located spinous process;
wherein the medial faces of both the first and second plates in both their superior and inferior end sections each have a plurality of protrusions configured to bite into the corresponding spinous process;
the second plate having a fastener bore and an anterior edge disposed opposite the fastener bore; the second plate having a channel therein opening at the anterior edge and extending posteriorly generally toward the fastener bore; the channel having a locking surface disposed posteriorly of the anterior edge and in spaced relation to the fastener bore;
the fastener bore opening anteriorly to the channel;
wherein the channel and the post are jointly configured such that the second plate may be moved over the post in an anterior direction normal to the post axis, while the post extends distally of the medial face of the second plate, to move the post from a position external to the second plate, posteriorly past the anterior edge of second plate, and into engagement with the locking surface;
wherein the fastener, post, and second plate are configured such that anterior advancement of the fastener, while the post abuts the locking surface, clamps the post between the fastener and the locking surface.

2. The spinal implant of claim 1 wherein the fastener is a setscrew.

3. The spinal implant of claim 1 wherein the fastener bore has a centerline; wherein the centerline does not pass through the locking surface.

4. The spinal implant of claim 1 wherein the post is pivotally mounted to the first plate.

5. The spinal implant of claim 1 wherein the second plate is infinitely positionable along the post axis relative to the first plate.

6. A spinal implant, comprising:
a first plate having a medial face configured to confront adjacent spinous processes;
a second plate having a medial face configured to confront the adjacent spinous processes; said second plate disposed in spaced relation to said first plate;
a post connecting the second plate to the first plate and extending distally from the first plate to a distal tip along a longitudinal post axis oriented transverse to the medial face of the second plate;
wherein the medial faces of each of the plates extend from the post in opposite directions therefrom between a superior end section of the respective plate that is positionable along a superiorly located spinous process and an inferior end section of the respective plate positionable along an inferiorly located spinous process; wherein the medial faces of both the first and second plates in both of their superior and inferior end sections each have a plurality of protrusions configured to bite into the corresponding spinous process;
the second plate having a fastener bore and an anterior edge opposite the fastener bore; the second plate having a channel therein defined by a boundary wall; the channel opening at the anterior edge and extending posteriorly generally toward the fastener bore; a portion of the boundary wall forming a posteriorly facing locking surface;
a fastener disposed in the fastener bore and movable relative to the second plate in a fastener advancement direction;
a first theoretical plane disposed normal to the post axis;
the post, from the medial face of the second plate to its distal tip, having a cross-section having a first size projected onto the first plane;
wherein the post is movable relative to the second plate, while extending through the channel such that the post tip is distally disposed relative to the medial face of the second plate and a centerline of the fastener bore, between a load position and a lock position;
wherein, with the post in the load position:
the post is spaced from the locking surface;
superior and inferior surfaces of the post directly face the boundary wall of the channel in unobstructed fashion;
a cross-section of the channel from the post to the anterior edge, projected onto the theoretical plane, does not narrow to less than the first size of the post;
wherein, with the post in the lock position:
the fastener supplies a force to clamp the post against the locking surface to affix the second plate to the post.

7. The spinal implant of claim 6 wherein the fastener is a setscrew.

8. The spinal implant of claim 6 wherein the centerline does not pass through the locking surface.

9. The spinal implant of claim 6 wherein the second plate is infinitely positionable along the post axis relative to the first plate.

10. A method of implanting a spinal implant, comprising:
positioning a first plate along a first lateral side of adjacent spinous processes; said positioning comprising disposing a post mounted to the first plate through the interspinous space between the adjacent spinous processes;
positioning a second plate posteriorly of the first plate with a distal tip of the post disposed distally of the medial face of the second plate; the second plate having a fastener bore and a channel therein disposed anteriorly of the fastener bore; the channel extending from an anterior edge of the second plate generally toward the fastener bore and having a locking surface;
thereafter, moving the second plate anteriorly relative to the post so that the post moves past the anterior edge and into the channel;
thereafter:
moving the second plate further anteriorly relative to the post so that the post abuts the locking surface;
moving the second plate along the post toward the first plate so that protrusions on medial faces of both the first and second plate bite into the adjacent spinous processes;
thereafter, tightening a locking member disposed in the fastener bore to supply a clamping force that clamps the post against the locking surface.

11. The method of claim 10 further comprising pivoting the post relative to the first plate.

12. The method of claim 10 wherein the distal tip of the post is disposed distally of the second plate during the moving the second plate anteriorly relative to the post so that the post moves past the anterior edge and into the channel.

13. The method of claim 10 wherein the moving the second plate so that the post abuts the locking surface comprises moving the second plate both anteriorly and superiorly relative to the post.

14. The method of claim 10 wherein the tightening the locking member comprises advancing the locking member anteriorly by rotating the locking member relative to the second plate.

15. The method of claim 10 wherein, at the conclusion of the tightening, the entirety of the post disposed distally of the medial face of the second plate is disposed posteriorly of the locking surface.
